(19) United States
(12) Patent Application Publication
(10) Pub. No.: US 2025/0230459 A1
(43) Pub. Date: Jul. 17, 2025

(54) METHOD FOR PRODUCING A GENETICALLY MODIFIED PLANT ORGANISM AND GENETICALLY MODIFIED ORGANISM OBTAINABLE THEREBY

(71) Applicants: TMCI PADOVAN S.P.A., Mareno Di Piave (TV) (IT); UNIVERSITA' DEGLI STUDI DI PADOVA, Padova (IT)

(72) Inventors: Tomas MOROSINOTTO, Padova (IT); Nicola MARON, Mareno Di Piave (TV) (IT); Diana SIMIONATO, Mareno Di Piave (TV) (IT); Francesca BUCCI, Padova (IT)

(73) Assignees: TMCI PADOVAN S.P.A., Mareno Di Paive (TV) (IT); UNIVERSITA' DEGLI STUDI DI PADOVA, Padova (IT)

(21) Appl. No.: 18/833,347

(22) PCT Filed: Jan. 25, 2023
(86) PCT No.: PCT/IT2023/050021

§ 371 Date: Jul. 25, 2024

(30) Foreign Application Priority Data
Jan. 25, 2022 (IT) 102022000001253

(51) Int. Cl. C12N 15/82; C12P 7/64

(52) U.S. Cl. CPC C12N 15/8247; C12P 7/64

(57) ABSTRACT

Method for producing a genetically modified plant organism, in particular algae or microalgae, which has the ability to accumulate lipids with a modified lipid profile for the purpose of producing oils for food use.

### CROSS-REFERENCE TO RELATED APPLICATIONS

This application is a U.S. National Stage Entry under 35 U.S.C. § 371 of International Patent Application No. PCT/IT2023/050021, entitled METHOD FOR PRODUCING A GENETICALLY MODIFIED PLANT ORGANISM AND GENETICALLY MODIFIED ORGANISM OBTAINABLE THEREBY, filed Jan. 25, 2023, which claims benefit of Italian Patent Application No. 102022000001253, filed Jan. 25, 2022, the entire disclosures of which are hereby incorporated by reference herein.

### FIELD OF THE INVENTION

Embodiments described here concern methods for producing genetically modified plant organisms and the genetically modified plant organisms obtainable thereby. In particular, the genetically modified plant organisms described here may be algae, more particularly microalgae. More particularly, embodiments described here concern the production of food oils (lipids) of microalgal origin which complement or replace the plant oils currently on the market. Compared to the production of other plant oils, such as for example olive oil, seed oil and palm oil, the production of oils from microalgae has advantages in terms of environmental sustainability, avoiding the exploitation of the soil caused by monoculture crops.

### BACKGROUND OF THE INVENTION

It is known that lipids are hydrophobic organic compounds and are divided into several classes. Among these there is the class of glycerides or acylglycerols, consisting of a molecule of glycerol to which one, two or three molecules of fatty acids (mono-, di-, triglycerides) are esterified. Food oils mainly consist of triglycerides (or triacylglycerols, TAGs); what changes between the various oils on the market is the length and degree of saturation of the fatty acid chains and their relative abundance.

To date it is known that microalgae in certain growth conditions accumulate large quantities of lipids, up to 70% of their dry weight. In particular, microalgae are single-celled algae that are individually invisible to the naked eye and are organisms able to accumulate lipids consisting of medium-long chain fatty acids: from 14 (C14) to 18 (C18) carbon atoms and 20 carbon atoms (C20) in some species, with variable degree of saturation of the fatty acid.

In order for oils of microalgal origin to be valid substitutes for the current plant oils on the market, it is necessary to reduce the content of medium-short chain fatty acids, up to C16, and to increase the content of saturated/unsaturated C18 and C20.

Document WO 2012/149457 A2 describes isolated nucleotide sequences that code polypeptides having a desaturase activity, which use fatty acids as substrates.

The document Janssen Jorijn H., et al. doi:10.1007/S10811-019-02021-2 describes the time-dependent transcriptome profile of genes involved in the synthesis of triacylglycerol (TAG) and polyunsaturated fatty acids in Nannochloropsis gaditana in the course of nitrogen deficiency. The gene expression of the genes involved in the lipid metabolism of the alga Nannochloropsis gaditana is measured by means of transcriptomic data. This microalga can be used as a source of TAG and omega-3 eicosapentaenoic fatty acid (EPA).

There is therefore a need to perfect microalgae which can overcome at least one of the disadvantages of the state of the art.

In particular, one purpose of the present invention is to obtain microalgae which accumulate lipids with a profile of fatty acids consisting of long chains, mainly C18.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the present invention or variants to the main inventive idea.

In accordance with some embodiments, there is provided a method for producing a genetically modified plant organism with a lipid profile which has a reduced content of C16 and an increased content of C18 compared to a control plant organism. In one embodiment, the method comprises the stable form gene silencing of a gene of the genome of the plant organism coding for the amino acid sequence defined by SEQ ID No. 1, wherein the gene is involved in the lipid biosynthesis pathway in the plant organism and consequently the silencing reduces the content of C16:1 and increases the content of C18:1.

In accordance with some embodiments, the biological function of the polypeptide having the sequence shown in SEQ ID No. 1 is that of a desaturase, the enzyme responsible for adding a double bond to the acyl chains. These enzymes are extremely precise, both in the choice of the substrate (that is, with a desired and defined chain length and number of double bonds) as well as in the addition of the double bond (position 6, 9, 12 and so on).

In accordance with some embodiments, the plant organism is an alga, in particular a microalga.

In accordance with some embodiments, the gene silencing can be carried out with any genetic engineering technique whatsoever, depending on the availability of approaches for the plant organism of interest.

In accordance with some embodiments, the gene silencing is carried out by means of DNA editing, in particular homologous recombination, or RNA interference.

In accordance with some embodiments, there is provided a vector in a plant organism.

In accordance with some possible embodiments, the vector is configured to determine the stable form gene silencing of a gene, wherein the sequence of the gene subject to silencing codes for the sequence defined by SEQ ID No. 1, wherein the gene is involved in the lipid biosynthesis pathway in the plant organism and consequently the silencing reduces the content of C16:1 and increases the content of C18:1. In accordance with some possible embodiments, the vector comprises two non-coding gene sequences present upstream and downstream of the gene subject to stable form silencing. The expression cassette for resistance to an antibiotic is cloned within these two regions. The coded amino acid sequence of the gene subject to silencing is defined by SEQ ID No. 1. The gene that is subjected to gene silencing is involved in the lipid biosynthesis pathway in the plant organism and consequently the silencing reduces the content of C16:1 and increases the content of C18:1.

In some embodiments described here, the vector is considered as only functional for the modification because it carries the gene sequences which allow the homologous recombination, therefore the elimination of the gene that codes the polypeptide (SEQ ID No. 1). The two regions for the homologous recombination include within them the cassette that gives the strain the resistance to the antibiotic (e.g. hygromycin; however, another antibiotic can be used). Furthermore, the antibiotic is only necessary for the isolation of the transformed strains, for which the modification will then have to be sought.

In accordance with some possible embodiments, there is provided a transformed clone of a plant organism containing a vector as disclosed heretofore.

In accordance with some embodiments, there is provided a genetically modified plant organism containing genomic DNA mutated by means of stable form gene silencing of a gene of the genome of the plant organism having a sequence that codes for the amino acid sequence defined by SEQ ID No. 1. The gene is involved in the lipid biosynthesis pathway in the plant organism and consequently the silencing reduces the content of C16:1 and increases the content of C18:1.

In some embodiments, the term stable form gene silencing can be understood to mean that the sequence SEQ ID No. 1 is no longer coded, since its gene has been replaced by the antibiotic resistance cassette.

In accordance with some embodiments, the plant organism is a microalga, more in particular belonging to the genus Nannochloropsis, in particular selected among the species N. gaditana, N. oceanica, N. salina, or belonging to the genus Thalassiosira, in particular T. pseudonana, or belonging to the genus Chlorella, in particular C. variabilis, or belonging to the genus Dunaliella, in particular D. salina.

In accordance with some embodiments, in the case of N. gaditana a gene having a sequence defined by SEQ ID No. 3 is silenced, in the case of N. oceanica a gene having a sequence defined by SEQ ID No. 5 is silenced, in the case of N. salina a gene having a sequence defined by SEQ ID No. 4 is silenced, in the case of T. pseudonana a gene having a sequence defined by SEQ ID No. 6 is silenced, in the case of C. variabilis a gene having a sequence defined by SEQ ID No. 7 is silenced, in the case of D. salina a gene that codes for a protein (polypeptide) having a sequence defined by SEQ ID No. 8 and/or SEQ ID No. 9 is silenced, in the case of P. tricornutum a gene having a sequence defined by SEQ ID No. 2 is silenced.

In accordance with some embodiments, there is provided a method for producing oils for food use, which comprises cultivating genetically modified plant organisms obtainable in accordance with the present description, so as to accumulate lipids with a profile that has a reduced content of C16:1 and an increased content of C18:1 compared to a control plant organism.

In accordance with some possible embodiments, there is provided a method for obtaining a genetically modified plant organism which provides to:
i) transform a cell culture or a plant or plant organism with a vector as described herein, obtaining transformed clones;
ii) select the transformed clones, in particular by means of selective medium, therefore with antibiotic;
iii) verify, by means of PCR (Polymerase Chain Reaction) technique, the occurred gene silencing in the one or more plant organisms obtained from ii), so that they show silencing of gene sequences involved in the lipid biosynthesis pathway.

### BRIEF DESCRIPTION OF THE DRAWINGS

FIG. 1 shows the homology regions of the Naga_100013g52 gene and inside the resistance cassette and the Naga_100013g52 gene, in accordance with the present invention;

FIG. 2 shows the verification by means of PCR of the absence of the transcript in the KO strain and the presence in the parental strain, in accordance with the present invention;

FIG. 3 shows a graph of the growth rate analysis between the parental strain and the KO, in accordance with the present invention;

FIG. 4 shows the lipid profile of the KO strain compared to the parental strain, in accordance with the present invention;

FIG. 5 shows the translated nucleotide sequences for eight proteins in accordance with the present invention; and

FIG. 6 shows the alignment of sequences in accordance with the present invention.

### DESCRIPTION OF SOME EMBODIMENTS

We shall now refer in detail to the various embodiments of the invention, of which one or more examples are shown in the attached drawings. Each example is supplied by way of illustration of the invention and shall not be understood as a limitation thereof. For example, the characteristics shown or described insomuch as they are part of one embodiment can be adopted on, or in association with, other embodiments to produce another embodiment. It is understood that the present invention shall include all such modifications and variants.

Before describing these embodiments, we must also clarify that the present description is not limited in its application to details of the construction and disposition of the components as described in the following description using the attached drawings. The present description can provide other embodiments and can be obtained or executed in various other ways. We must also clarify that the phraseology and terminology used here is for the purposes of description only, and cannot be considered as limitative.

Unless otherwise defined, all the technical and scientific terms used here and hereafter have the same meaning as commonly understood by a person with ordinary experience in the field of the art to which the present invention belongs. Even if methods and materials similar or equivalent to those described here can be used in practice and in the trials of the present invention, the methods and materials are described hereafter as an example. In the event of conflict, the present application shall prevail, including its definitions. The materials, methods and examples have a purely illustrative purpose and shall not be understood restrictively.

All measurements are carried out at 25° C. and at atmospheric pressure, unless otherwise indicated. All temperatures are expressed in degrees Celsius, unless otherwise indicated.

Some embodiments described here concern the production of food oils (lipids) of microalgal origin which complement/replace the plant oils currently on the market. Compared to the production of other plant oils (e.g. olive oil, seed oil, palm oil, etc.), the production of oils from microalgae offers advantages in terms of environmental sustainability, avoiding the exploitation of the soil caused by monoculture crops.

In particular, the Applicant has developed microalgae that accumulate lipids with a fatty acid profile consisting of long chains, mainly C18:1, while the C16:1 content is reduced. The notations C16:1 and C18:1 indicate the presence of a double bond in the fatty acid. This fatty acid profile is obtained by silencing gene sequences involved in the lipid biosynthesis pathway in the microalgae.

Through the gene silencing of sequences involved in lipid biosynthesis it is therefore possible to increase the content of long-chain oils in various species of microalgae, so that they can be exploited in the food sector.

Such gene silencing can be performed with any genetic engineering technique, depending on the availability of approaches for the microalga of interest.

The Applicant has therefore found that targeted genetic modifications of sequences involved in the lipid biosynthesis can reduce the content of short-chain fatty acids in favor of those with longer chains.

### Example of Gene Silencing in Nannochloropsis gaditana

The Applicant analyzed the biosynthesis pathway of the fatty acids of the microalga Nannochloropsis gaditana and identified the Naga_100013g52 gene as a target for gene silencing—SEQ ID No. 3. To carry out this silencing, it was decided to replace the gene by homologous recombination with the hygromycin resistance cassette. Homologous recombination is a DNA repair mechanism that occurs with different frequencies and efficiencies depending on the organism examined. In the case of Nannochloropsis gaditana, it is an extremely rare phenomenon (less than 1%), therefore the CRISPR-Cas technique was used to increase its efficiency. This system is based on the use of proteins (Cas9 being the most well-known; for this experiment Cpf1 was used) with precise nucleolytic activity. In fact, they bind RNA molecules, called guide RNAs (gRNAs) of about 20 nucleotides, which recognize DNA in a specific, complementary way, thus directing the cleavage by the Cas. The Cas protein and gRNA complex can be referred to as a ribonucleoprotein. Furthermore, in order for the ribonucleoprotein to bind to the DNA, the protein has to recognize on it a sequence defined as PAM (Protospacer Adjacent Motif).

In this experiment, the Cpf1 protein was chosen because the PAM sequences it recognizes in the N. gaditana genome are larger than those recognized by Cas9. Three different guides were used to target the endonucleolytic cleavage of the DNA by Cpf1. These guides were chosen with the online tool CRISPOR (http://crispor.tefor.net/), which elaborates a detailed analysis of the genome so that the identified guides do not also recognize off-target sequences. The three guides cover different regions of the gene, starting from the 5′ to the 3′. The guides selected are the following:

1. 5′-GTAGTGCTTGTCGGAGACATGGT-3′ indicated as
SEQ ID No. 10
2. 5′-AAGGAGATCTTGAACAGAGACCC-3′ indicated as
SEQ ID No. 11
3. 5′-TTGATAACGTGCCCCCTCGCCAG-3′ indicated as
SEQ ID No. 12

There are organisms in which the homologous recombination is more frequent, and it is not necessary to induce it with the cleavage of the DNA with the CRISPR-Cas system.

Furthermore, in addition to silencing by homologous recombination, or so-called DNA editing approaches in general, there are techniques based on RNA interference (RNAi), in which RNA molecules interfere with gene expression, for example by degrading messenger RNA (mRNA). Protocols of this type have not yet been developed for N. gaditana; moreover, it is known that these may be transient silencings, therefore to be repeated over time in order to obtain the desired strain.

A culture of N. gaditana (strain CCAP 849/5) grown for 5 days, starting from 7.10⁶ cells/ml in a 250 ml bottle in F/2 medium (salinity 32 g/L, 40 mM Tris-HCl, pH8) supplemented with 12 mM NaNO₃ and 5% CO₂ (v/v), was transformed by electroporation (protocol specifications: voltage: 2000 V; resistance: 600Ω; capacity: 50 μF) with 5 μg of linearized plasmid DNA and ribonucleoproteins (6 μM of each guide and 8 μg of purified Cpf1 (IDT)). The plasmid DNA consists of the hygromycin (antibiotic) resistance cassette comprised upstream and downstream of 1000 bp homologous to the sequences upstream and downstream of the Naga_100013g52 sequence. This construction allowed the phenomenon of homologous recombination, such that the Naga_100013g52 gene was replaced by the hygromycin resistance cassette.

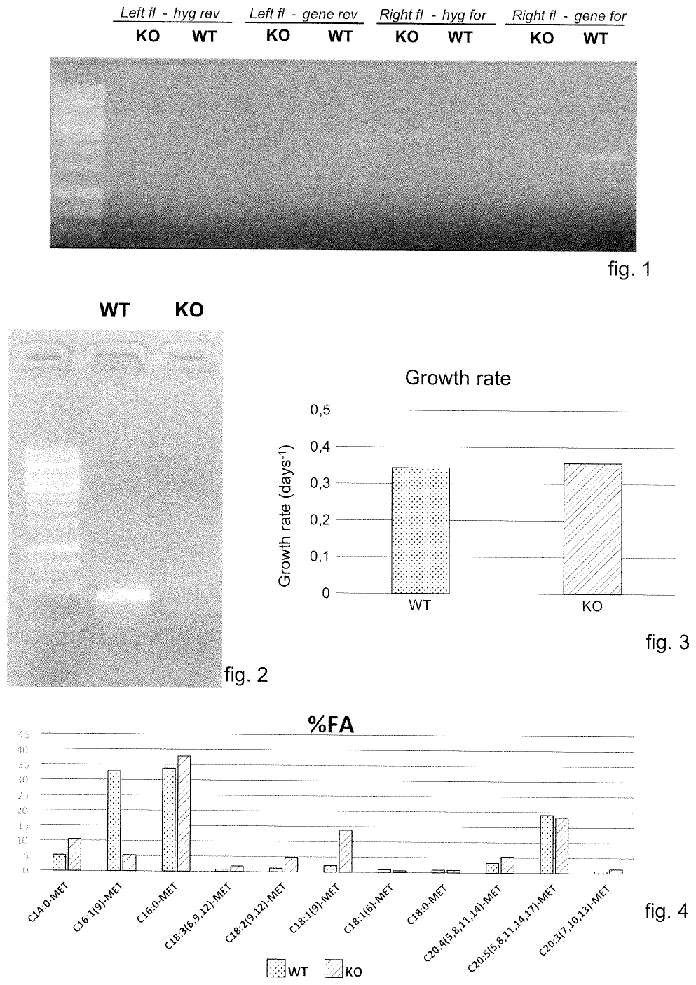

The selection of the transformed strains occurred on selective medium (supplemented with 500 μg/ml of hygromycin). Subsequently, the homologous recombination event, that is, the replacement of the Naga_100013g52 gene with the hygromycin resistance cassette, was verified by means of PCR on genomic DNA of the mutant strain (Knock-Out KO) of N. gaditana. The primers used for the PCR reaction were drawn outside the homology regions of the Naga_100013g52 gene (Left fl and Right fl in FIG. 1) and inside the resistance cassette (hyg for and hyg rev) and the Naga_100013g52 gene (gene for and gene rev). The homologous recombination efficiency recorded is extremely low: less than 1%, therefore only one strain was examined. The primer sequences are shown below:

Left fl:
5′-TCGACCGCTCCATGTTGCAA-3′ indicated as
SEQ ID No. 13
Gene for:
5′-CTTACAAGGGCTTCGTCTAC-3′indicated as
SEQ ID No. 14
Gene rev:
5′-AGCTTTTCTTGATAACGTGC-3′ indicated as
SEQ ID No. 15
Right fl:
5′-GTCATCGTCGTATCCGAGAG-3′ indicated as
SEQ ID No. 16
Hyg for:
5′-ATGAAAAAGCCTGAACTCAC-3′ indicated as
SEQ ID No. 17
Hyg rev:
5′-CTATTCCTTTGCCCTCGGAC-3′ indicated as
SEQ ID No. 18.

The mRNA was also extracted, and reverse transcribed into cDNA to verify the expression of the Naga_100013g52 gene. The absence of the transcript in the KO strain, present instead in the parental strain (Wild Type—WT), was verified by means of PCR with the primers RNA for and RNA rev, as shown in FIG. 2. The primer sequences are shown below:

RNA for:
5′-CTTACAAGGGCTTCGTCTAC-3′ indicated as
SEQ ID No. 19
RNA rev:
5′-AGCTTTTCTTGATAACGTGC-3′ indicated as
SEQ ID No. 20.

### Comparative Experimental Data in Nannochloropsis gaditana

A KO strain was obtained by silencing the Naga_100013g52 sequence of Nannochloropsis gaditana.

Two cultures were prepared for comparison, one of the parental strain (WT) of N. gaditana and the other of the KO strain, both kept in semi-continuous mode in 250 ml bottles supplemented with CO₂ at 5% (v/v), in F/2 medium (salinity 32 g/L, Tris-HCl 40 mM, pH8) rich in nutrients (0.75 g/L NaNO₃, 0.05 g/L NaH₂PO₄ and 0.0063 g/L FeCl₃·6 H₂O final concentration). The cultures were diluted with fresh medium every two/three days in order to bring the cell concentration back to 150·10⁶ cells/ml. This mode allowed for growth in optimal conditions, keeping the microalgae in active exponential growth. From the analysis of the growth rate, no differences were reported between the parental strain and the KO (graph of FIG. 3).

At each dilution of the cultures, the excess material, deprived of the growth liquid, was stored at a temperature of −80° C. and subsequently lyophilized to finally carry out a lipidomic analysis. This consists in the extraction of total lipids in the form of FAME (Fatty Acid Methyl Esthers), then quantified by means of Gas-Chromatography. Each detected signal was assigned to a specific fatty acid class using a mass spectrometer. A different lipid profile was then found for the KO strain (shown with a diagonal line hatching in FIG. 4) compared to the parental strain (WT).

The KO strain is characterized by a six-fold reduction in the content of C16:1 and a seven-fold increase in C18:1.

### Extension to Other Microalgae

To extend the KO approach of the same gene in other microalgae, in order to obtain oils for food use, homologous genes were sought in other species besides N. gaditana. The experiment involved the use of the software BLAST (Basic Local Alignment Search Tool) with standard search parameters.

From the analysis of Naga_100013g52 sequence homology in other species of microalgae, other sequences were identified, annotated as genes or not (in other cases, the proteins were identified from which the coding sequence was then traced), listed below:
Nannochloropsis oceanica LOCUS CP044584.1:437997-439297—SEQ ID No. 5
Nannochloropsis salina scaffold_5:444671-445933—SEQ ID No. 4
Thalassiosira pseudonana ACCESSION N. GENE XM_002297328.1; PROTEIN XP_002297364.1—SEQ ID No. 6
Dunaliella salina PROTEIN 1 KAF5835254.1—SEQ ID No. 8; PROTEIN 2 KAF5838958.1—SEQ ID No. 9
Chlorella variabilis ACCESSION N. GENE XM_005848291.1; PROTEIN XP_005848353.1—SEQ ID No. 7
Phaeodactylum tricornutum ACCESSION N. GENE KT160314; PROTEIN ANQ45194.1—SEQ ID No. 2

From the gene silencing of these sequences, the Applicant expects a lipid profile with reduced C16:1 (palmitoleic acid) and increased C18:1 (oleic acid) content.

These gene sequences were translated and then aligned using the online tool clustal omega (https://www.ebi.ac.uk/Tools/msa/clustalo/) available on the EMBL-EBI website. Alignment is a bioinformatics procedure that allows to compare two or more sequences in order to identify conserved residues and/or regions, plausibly characteristic of the proteins.

Alignments can also be made with nucleotide sequences, but they are less informative due to the fact that the genetic code is redundant and different nucleotide triplets can code for the same amino acid.

The N-terminal region (therefore the first part of the protein) is species specific and directs the protein to the correct cellular compartment. For this reason, these regions may not be conserved and only the subsequent residues will be taken into consideration. Furthermore, the alignment showed that the residues at the C-terminal are equally poorly conserved.

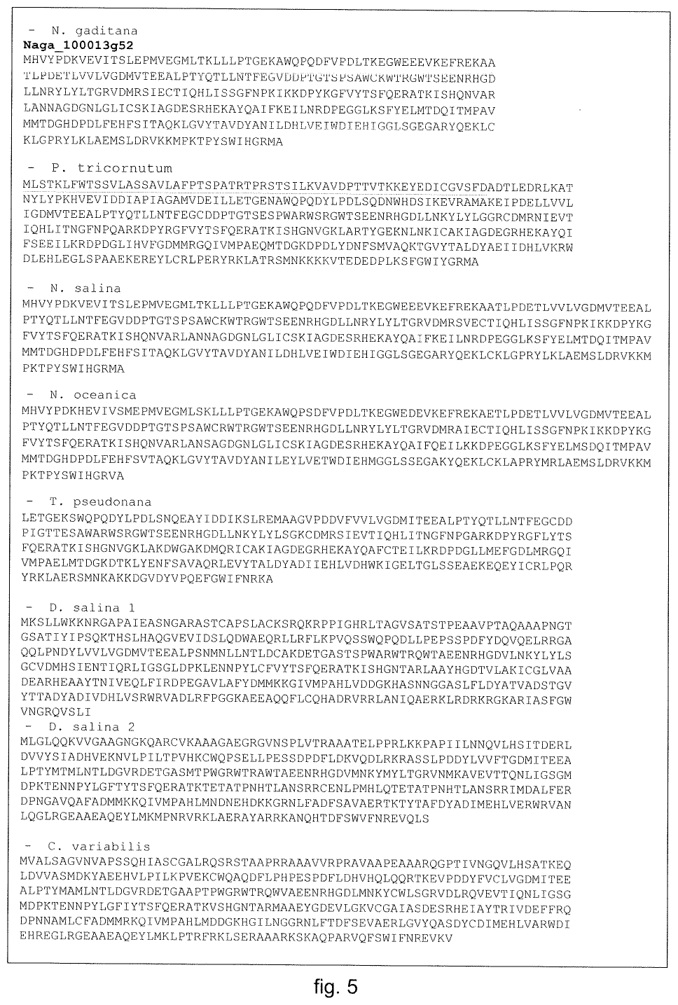

FIG. 5 shows the translated nucleotide sequences for the eight proteins identified above:
N. gaditana—SEQ ID No. 3 and respectively SEQ ID No. 21
P. tricornutum—SEQ ID No. 2 and respectively SEQ ID No. 22
N. salina—SEQ ID No. 4 and respectively SEQ ID No. 23
N. oceanica—SEQ ID No. 5 and respectively SEQ ID No. 24
T. pseudonana—SEQ ID No. 6 and respectively SEQ ID No. 25
D. salina 1—SEQ ID No. 8
D. salina 2—SEQ ID No. 9
C. variabilis—SEQ ID No. 7 and respectively SEQ ID No. 26.

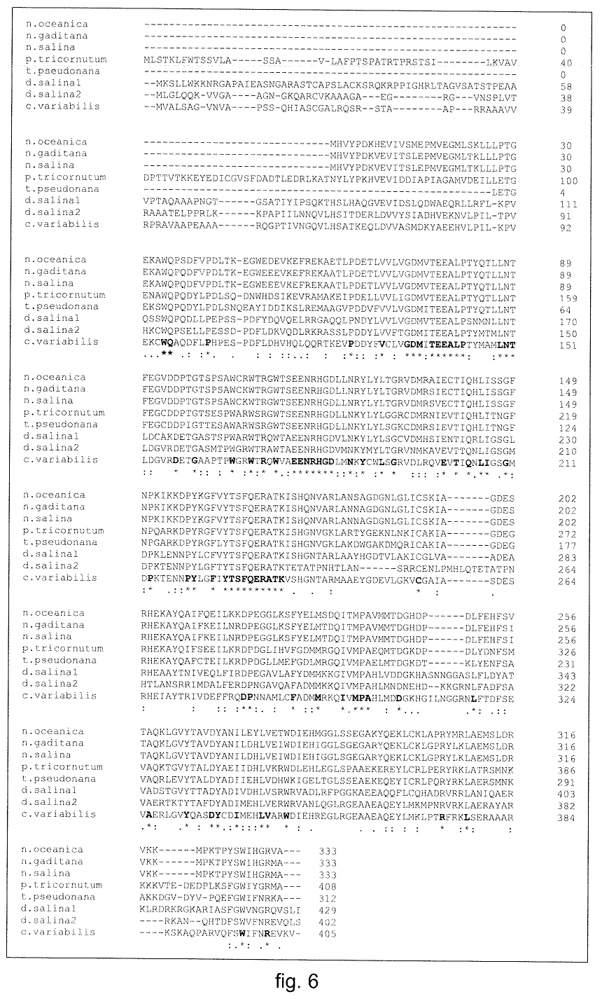

FIG. 6 shows the alignment of the sequences as above.

From the analysis of the alignment of the protein sequences, the following profile of conserved residues was obtained, as defined by the sequence of the profile of the residues conserved in SEQ ID No. 1. Below, the conserved residues are written with the letter corresponding to the amino acid, the possible residues for that position are identified in brackets and finally X indicates that any residue can be found.

P [D, N] [D, E] [T, L, V, Y] [L, F] V [V, C]
[L, F] [V, I, T] GDM [V, I] TEEALP [T, S] [Y, N]
[Q, M] [T, N, A] [L, M] LNT [F, L] [E, D] [G, C]
[V, C, A] [D, K, R] D [P, E] [T, I] G [A, T]
[A, T, S] X [S, T] [A, P] W [A, C, G] [K, R] W
[T, S] RXW [T, V] [S, A] EENRHGD [L, V] [L, M] N
[R, K] Y [L, M, C] [Y, W] L [T, G, S] G X [C, V]
[D, N] [M, L] [R, H, K] X [I, V] E [C, V, N] T
[I, T] Q [H, R, N] LI [S, T, G] [S, N] G [F, L,
M] [D, N] P XX [K, R, E] [K, N] [D, N] PY [K,
R, L] [G, C] F X YTSFQERATK [I, T, V] [S, E] [H,
T] [Q, G, A] [N, T] [V, T, P] [A, G, N] [R, K, H]
[L, T, M] [A, L] XXXXXXXXXXX C XX [I, V, L] [A, P]
[G, A, S] [D, T] [E, P] X [R, H] [H, T] [E, L] [K,
A, I] [A, N] [S, Y] [Q, T, R] X [I, F] XX [Q, A,
E] [I, L, F] [L, F] X [K, R, Q] DP [D, E, N] [G,
N] XXXX F [G, Y, A] [E, D] [L, M] M X [D, G, K]
[Q, G] I [T, V] MPA [V, E, H] [M, Q, L] [V, M]
[T, D, N] D [G, N] [H, K, E] [D, H] XX L [Y, F] X
[H, N, D][Y, F] [A, S] X [T, V] A [Q, D, E] [K, R,
S] [L, T] [G, K] [T, V] Y [T, Q] [A, T] X DY [A,
C] [N, E, D] I X [E, D] [H, Y] LV XX W

In particular, with regard to the above mentioned sequences SEQ ID No: 3-7, we must specify that first the gene sequences were identified, then they were translated in order to have the proteins; these were aligned and then the final sequence with the conserved residues (SEQ ID No. 1) was generated. Obviously, in practice the proteins have different lengths so the software only aligns the conserved portions and, therefore, it may be that some residues are cut out; these were not considered in the alignment, since there would have been no way to do this. However, this does not negatively affect the present analysis.

The alignment therefore produced a highly conserved profile (except for the regions at the N and C terminal which are more species specific). Even if the uniqueness of residue is found for few amino acids, it is often possible to find, as an alternative, only two or three amino acids, mostly with the same chemical characteristics. Please note, for example, that E (glutamate) and D (aspartate) are two polar amino acids (negatively charged) and therefore replacing one with the other leads to changes in the primary structure of the protein, but not in the secondary and tertiary one, that is, in the three-dimensional and functional structure of the protein.

The presence of these conserved domains is presumably necessary not only for the correct formation of alpha helix structures, of which these proteins are rich, but also for the interactions that the proteins can have with their substrates. In fact, considering the phenotype observed in the case of gene silencing of the sequences coding for these proteins, it is reasonable to think that they are necessary for the interaction with acyl chains of 16 carbon atoms.

The Applicant therefore believes that it is plausibly possible to obtain an accumulation of lipids with a profile that has a reduced content of C16:1 and an increased content of C18:1, compared to a control plant organism, in a genetically modified plant organism containing genomic DNA mutated in a stable form by gene silencing of a gene coding an amino acid sequence defined by SEQ ID No. 1 of the genome of such plant organism, in which such gene is involved in the lipid biosynthesis pathway in such plant organism and consequently the silencing as above reduces the content of C16:1 and increases the content of C18:1.

The Applicant, by virtue of the fact that the degree of conservation is high, deems it plausible that the polypeptide sequence conserved in the organisms described in the present description has the same function, and in some cases (e.g.: Phaeodactylum tricornutum) this has been demonstrated.

Moreover, the Applicant deems it plausible that the polypeptide has the same phenotype (effect) in the plant organisms mentioned herein and in particular in microalgae. The Applicant, on the basis of the experimental evidence presented, also believes that in all likelihood there are also other plants which conserve this sequence.

The silencing described here saw the use of the CRISPR-Cas system and, in possible embodiments, replacement of the gene with a cassette for resistance to an antibiotic (hygromycin) through homologous recombination. The Applicant believes that this is a permanent modification, considering that the strain under examination was isolated for experimental purposes in January 2021 and has always maintained the modification to date. Due to the nature of the modification, since a real replacement of DNA sequences has occurred, this cannot be reversible but, on the contrary, the Applicant expects it to be maintained stably.

It is clear that modifications and/or additions of parts may be made to the method and to the modified plant organisms as described heretofore, without departing from the field and scope of the present invention, as defined by the claims. It is also clear that, although the present invention has been described with reference to some specific examples, a person of skill in the art shall certainly be able to achieve many other equivalent forms of method and modified plant organisms, having the characteristics as set forth in the claims and hence all coming within the field of protection defined thereby.

1. A method for producing a genetically modified plant organism with a lipid profile which has a reduced content of C16:1 and an increased content of C18:1 compared to a control plant organism, said method comprising stable form gene silencing of a gene of the genome of said plant organism coding for an amino acid sequence defined by SEQ ID No. 1, wherein said gene is involved in an lipid biosynthesis pathway in said plant organism and consequently said silencing reduces the content of C16:1 and increases the content of C18:1.

2. The method of claim 1, wherein said plant organism is an alga, in particular a microalga.

3. The method of claim 1, wherein said gene silencing is carried out by means of DNA editing, in particular homologous recombination, or RNA interference.

4. A vector in a plant organism configured to determine a stable form gene silencing of a gene, wherein a sequence of said gene subject to silencing codes for an amino acid sequence defined by SEQ ID No. 1, wherein said gene is involved in a lipid biosynthesis pathway in said plant organism and consequently said silencing reduces a content of C16:1 and increases a content of C18:1.

5. A vector in a plant organism, comprising two non-coding gene sequences present upstream and downstream of a gene subject to stable form gene silencing, wherein an expression cassette for resistance to an antibiotic is cloned within these two regions, wherein the sequence of said gene subject to silencing codes for an amino acid sequence defined by SEQ ID No. 1, wherein said gene is involved in a lipid biosynthesis pathway in said plant organism and consequently said silencing reduces a content of C16:1 and increases a content of C18:1.

6. A transformed clone of a plant organism containing the vector of claim 4.

7. A genetically modified plant organism containing genomic DNA mutated by means of stable form gene silencing of a gene of a genome of said plant organism that codes for an amino acid sequence defined by SEQ ID No. 1, wherein said gene is involved in a lipid biosynthesis pathway in said plant organism and consequently said silencing reduces a content of C16:1 and increases a content of C18:1.

8. The plant organism of claim 7, wherein said plant organism is an alga, in particular a microalga, more in particular belonging to the genus Nannochloropsis, in particular selected among the species N. gaditana, N. oceanica, N. salina, or belonging to the genus Thalassiosira, in particular T. pseudonana, or belonging to the genus Chlorella, in particular C. variabilis, or belonging to the genus Dunaliella, in particular D. salina, or belonging to the genus Phaeodactylum, in particular P. tricornutum.

9. The plant organism of claim 8, wherein in the case of N. gaditana a gene having a sequence defined by SEQ ID No. 3 is silenced, in the case of N. oceanica a gene having a sequence defined by SEQ ID No. 5 is silenced, in the case of N. salina a gene having a sequence defined by SEQ ID No. 4 is silenced, in the case of T. pseudonana a gene having a sequence defined by SEQ ID No. 6 is silenced, in the case of C. variabilis a gene having a sequence defined by SEQ ID No. 7 is silenced, in the case of D. salina a gene that codes for a protein having a sequence defined by SEQ ID No. 8 and/or SEQ ID No. 9 is silenced, in the case of P. tricornutum a gene having a sequence defined by SEQ ID No. 2 is silenced.

10. A method for producing oils for food use, said method comprising cultivating genetically modified plant organisms obtainable in accordance with claim 1, so as to accumulate lipids with a profile that has a reduced content of C16:1 and an increased content of C18:1 compared to a control plant organism.

11. A transformed clone of a plant organism containing the vector of claim 5.

12. A method for producing oils for food use, said method comprising cultivating genetically modified plant organisms obtainable in accordance with claim 2, so as to accumulate lipids with a profile that has a reduced content of C16:1 and an increased content of C18:1 compared to a control plant organism.

13. A method for producing oils for food use, said method comprising cultivating genetically modified plant organisms obtainable in accordance with claim 3, so as to accumulate lipids with a profile that has a reduced content of C16:1 and an increased content of C18:1 compared to a control plant organism.

14. A method for producing oils for food use, said method comprising cultivating genetically modified plant organisms obtainable in accordance with claim 1, so as to accumulate lipids with a profile that has a reduced content of C16:1 and an increased content of C18:1 compared to a control plant organism.

15. A method for producing oils for food use, said method comprising cultivating genetically modified plant organisms in accordance with claim 7, so as to accumulate lipids with a profile that has a reduced content of C16:1 and an increased content of C18:1 compared to a control plant organism.

16. A method for producing oils for food use, said method comprising cultivating genetically modified plant organisms in accordance with claim 8, so as to accumulate lipids with a profile that has a reduced content of C16:1 and an increased content of C18:1 compared to a control plant organism.

17. A method for producing oils for food use, said method comprising cultivating genetically modified plant organisms in accordance with claim 9, so as to accumulate lipids with a profile that has a reduced content of C16:1 and an increased content of C18:1 compared to a control plant organism.


SEQUENCE LISTING (26 sequences)

SEQ ID NO: 1 - protein, 218 aa, synthetic construct
  REGION 1..218: note = Sequenza profilo residui conservati
  VARIANT 2: note = Xaa replaces Asp or Asn
  VARIANT 3: note = Xaa replaces Asp or Glu
  VARIANT 4: note = Xaa replaces Thr or Leu or Val or Thr
  VARIANT 5: note = Xaa replaces Leu or Phe
  VARIANT 7: note = Xaa replaces Val or Cys
  VARIANT 8: note = Xaa replace Leu or Phe
  VARIANT 9: note = Xaa replaces Val or IIe or Thr
  VARIANT 13: note = Xaa replaces Val or lle
  VARIANT 20: note = Xaa replaces Thr or Ser
  VARIANT 21: note = Xaa replaces Tyr or Asn
  VARIANT 22: note = Xaa replaces Gln or Met
  VARIANT 23: note = Xaa replaces Thr or Asn or Ala
  VARIANT 24: note = Xaa replaces Leu or Met
  VARIANT 28: note = Xaa replaces Phe or Leu
  VARIANT 29: note = Xaa replaces Glu or Asp
  VARIANT 30: note = Xaa replaces Gly or Cys
  VARIANT 31: note = Xaa replaces Val or Cys or Ala
  VARIANT 32: note = Xaa replaces Asp or Lys or Arg
  VARIANT 34: note = Xaa replaces Pro or Glu
  VARIANT 35: note = Xaa replaces Thr or lle
  VARIANT 37: note = Xaa replaces Ala or Thr
  VARIANT 38: note = Xaa replaces Ser or Thr or Ala
  VARIANT 39: note = Xaa replaces any amino acid
  VARIANT 40: note = Xaa replaces Ser or Thr
  VARIANT 41: note = Xaa replaces Ala or Pro
  VARIANT 43: note = Xaa replaces Ala or Cys or Gly
  VARIANT 44: note = Xaa replaces Lys or Arg
  VARIANT 46: note = Xaa replaces Thr or Ser
  VARIANT 48: note = Xaa replaces any amino acid
  VARIANT 50: note = Xaa replaces Thr or Val
  VARIANT 51: note = Xaa replaces Ser or Ala
  VARIANT 59: note = Xaa replaces Leu or Val
  VARIANT 60: note = Xaa replaces Leu or Met
  VARIANT 62: note = Xaa replaces Arg or Lys
  VARIANT 64: note = Xaa replaces Leu or Met or Cys
  VARIANT 65: note = Xaa replaces Tyr or Trp
  VARIANT 67: note = Xaa replaces Thr or Gly or Ser
  VARIANT 69: note = Xaa replaces any amino acid
  VARIANT 70: note = Xaa replaces Cys or Val
  VARIANT 71: note = Xaa replaces Asp or Asn
  VARIANT 72: note = Xaa replaces Met or Leu
  VARIANT 73: note = Xaa replaces Arg or His or Lys
  VARIANT 74: note = Xaa replaces any amino acid
  VARIANT 75: note = Xaa replaces lle or Val
  VARIANT 77: note = Xaa replaces Cys or Val or Asn
  VARIANT 79: note = Xaa replaces lle or Thr
  VARIANT 81: note = Xaa replaces His or Arg or Asn
  VARIANT 84: note = Xaa replaces Ser or Thr or Gly
  VARIANT 85: note = Xaa replaces Ser or Asn
  VARIANT 87: note = Xaa replaces Phe or Leu or Met
  VARIANT 88: note = Xaa replaces Asp or Asn
  VARIANT 90: note = Xaa replaces any amino acid
  VARIANT 91: note = Xaa replaces any amino acid
  VARIANT 92: note = Xaa replaces Lys or Arg or Glu
  VARIANT 93: note = Xaa replaces Lys or Asn
  VARIANT 94: note = Xaa replaces Asp or Asn
  VARIANT 97: note = Xaa replaces Lys or Arg or Leu
  VARIANT 98: note = Xaa replaces Gly or Cys
  VARIANT 100: note = Xaa replaces any amino acid
  VARIANT 111: note = Xaa replaces lle or Thr or Val
  VARIANT 112: note = Xaa replaces Ser or Glu
  VARIANT 113: note = Xaa replaces His or Thr
  VARIANT 114: note = Xaa replaces Gln or Gly or Ala
  VARIANT 115: note = Xaa replaces Asn or Thr
  VARIANT 116: note = Xaa replaces Val or Thr or Pro
  VARIANT 117: note = Xaa replaces Ala or Gly or Asn
  VARIANT 118: note = Xaa replaces Arg or Lys or His
  VARIANT 119: note = Xaa replaces Leu or Thr or Met
  VARIANT 120: note = Xaa replaces Ala or Leu
  VARIANT 121: note = Xaa replaces any amino acid
  VARIANT 122: note = Xaa replaces any amino acid
  VARIANT 123: note = Xaa replaces any amino acid
  VARIANT 124: note = Xaa replaces any amino acid
  VARIANT 125: note = Xaa replaces any amino acid
  VARIANT 126: note = Xaa replaces any amino acid
  VARIANT 127: note = Xaa replaces any amino acid
  VARIANT 128: note = Xaa replaces any amino acid
  VARIANT 129: note = Xaa replaces any amino acid
  VARIANT 130: note = Xaa replaces any amino acid
  VARIANT 131: note = Xaa replaces any amino acid
  VARIANT 133: note = Xaa replaces any amino acid
  VARIANT 134: note = Xaa replaces any amino acid
  VARIANT 135: note = Xaa replaces lle or Val or Leu
  VARIANT 136: note = Xaa replaces Ala or Pro
  VARIANT 137: note = Xaa replaces Gly or Ala or Ser
  VARIANT 138: note = Xaa replaces Asp or Thr
  VARIANT 139: note = Xaa replaces Glu or Pro
  VARIANT 140: note = Xaa replaces any amino acid
  VARIANT 141: note = Xaa replaces Arg or His
  VARIANT 142: note = Xaa replaces His or Thr
  VARIANT 143: note = Xaa replaces Glu or Leu
  VARIANT 144: note = Xaa replaces Lys or Ala or Ile
  VARIANT 145: note = Xaa replaces Ala or Asn
  VARIANT 146: note = Xaa replaces Ser or Tyr
  VARIANT 147: note = Xaa replaces Gln or Thr or Arg
  VARIANT 148: note = Xaa replaces any amino acid
  VARIANT 149: note = Xaa replaces lle or Phe
  VARIANT 150: note = Xaa replaces any amino acid
  VARIANT 151: note = Xaa replaces any amino acid
  VARIANT 152: note = Xaa replaces Gln or Ala or Glu
  VARIANT 153: note = Xaa replaces lle or Leu or Phe
  VARIANT 154: note = Xaa replaces Leu or Phe
  VARIANT 155: note = Xaa replaces any amino acid
  VARIANT 156: note = Xaa replaces Lys or Arg or Gln
  VARIANT 159: note = Xaa replaces Asp or Glu or Asn
  VARIANT 160: note = Xaa replaces Gly or Asn
  VARIANT 161: note = Xaa replaces Gly or Leu or Ala
  VARIANT 162: note = Xaa replaces any amino acid
  VARIANT 163: note = Xaa replaces any amino acid
  VARIANT 164: note = Xaa replaces any amino acid
  VARIANT 166: note = Xaa replaces Gly or Tyr or Ala
  VARIANT 167: note = Xaa replaces Glu or Asp
  VARIANT 168: note = Xaa replaces Leu or Met
  VARIANT 170: note = Xaa replaces any amino acid
  VARIANT 171: note = Xaa replaces Asp or Gly or Lys
  VARIANT 172: note = Xaa replaces Gln or Gly
  VARIANT 174: note = Xaa replaces Thr or Val
  VARIANT 178: note = Xaa replaces Val or Glu or His
  VARIANT 179: note = Xaa replaces Met or Gln or Leu
  VARIANT 180: note = Xaa replaces Val or Met
  VARIANT 181: note = Xaa replaces Thr or Asp or Asn
  VARIANT 183: note = Xaa replaces Gly or Asn
  VARIANT 184: note = Xaa replaces His or Lys or Glu
  VARIANT 185: note = Xaa replaces Asp or His
  VARIANT 186: note = Xaa replaces any amino acid
  VARIANT 187: note = Xaa replaces any amino acid
  VARIANT 189: note = Xaa replaces Tyr or Phe
  VARIANT 190: note = Xaa replaces any amino acid
  VARIANT 191: note = Xaa replaces His or Asn or Asp
  VARIANT 192: note = Xaa replaces Tyr or Phe
  VARIANT 193: note = Xaa replaces Ala or Ser
  VARIANT 194: note = Xaa replaces any amino acid
  VARIANT 195: note = Xaa replaces Thr or Val
  VARIANT 197: note = Xaa replaces Gln or Asp or Glu
  VARIANT 198: note = Xaa replaces Lys or Arg or Ser
  VARIANT 199: note = Xaa replaces Leu or Thr
  VARIANT 200: note = Xaa replaces Gly or Lys
  VARIANT 201: note = Xaa replaces Thr or Val
  VARIANT 203: note = Xaa replaces Thr or Gln
  VARIANT 204: note = Xaa replaces Ala or Thr
  VARIANT 205: note = Xaa replaces any amino acid
  VARIANT 208: note = Xaa replaces Ala or Cys
  VARIANT 209: note = Xaa replaces Asn or Glu or Asp
  VARIANT 211: note = Xaa replaces any amino acid
  VARIANT 212: note = Xaa replaces Glu or Asp
  VARIANT 213: note = Xaa replaces His or Tyr
  VARIANT 216: note = Xaa replaces any amino acid
  VARIANT 217: note = Xaa replaces any amino acid
PXXXXVXXXGDMXTEEALPXXXXXLNTXXXXXDXXGXXXXXWXXWXRXWXXEENRHGDXXNXYXXLXGXXXXXXXEXTXQXLIXXGXXPXXXXXPYXXFXYTSFQERATKXXXXXXXXXXXXXXXXXXXXXCXXXXXXXXXXXXXXXXXXXXXXXXDPXXXXXXFXXXMXXXIXMPAXXXXDXXXXXLXXXXXXXAXXXXXYXXXDYXXIXXXLVXXW

SEQ ID NO: 2 - RNA, 1227 nt, Phaeodactylum tricornutum
  modified_base 2: mod_base = OTHER; note = thymine
  modified_base 4..5: mod_base = OTHER; note = thymine
  modified_base 7: mod_base = OTHER; note = thymine
  modified_base 9: mod_base = OTHER; note = thymine
  modified_base 17..20: mod_base = OTHER; note = thymine
  modified_base 22: mod_base = OTHER; note = thymine
  modified_base 30..31: mod_base = OTHER; note = thymine
  modified_base 35..38: mod_base = OTHER; note = thymine
  modified_base 42..43: mod_base = OTHER; note = thymine
  modified_base 48: mod_base = OTHER; note = thymine
  modified_base 53: mod_base = OTHER; note = thymine
  modified_base 56..57: mod_base = OTHER; note = thymine
  modified_base 61..63: mod_base = OTHER; note = thymine
  modified_base 70: mod_base = OTHER; note = thymine
  modified_base 75: mod_base = OTHER; note = thymine
  modified_base 81: mod_base = OTHER; note = thymine
  modified_base 87: mod_base = OTHER; note = thymine
  modified_base 90: mod_base = OTHER; note = thymine
  modified_base 99..100: mod_base = OTHER; note = thymine
  modified_base 104..105: mod_base = OTHER; note = thymine
  modified_base 107: mod_base = OTHER; note = thymine
  modified_base 113..114: mod_base = OTHER; note = thymine
  modified_base 119: mod_base = OTHER; note = thymine
  modified_base 134: mod_base = OTHER; note = thymine
  modified_base 148: mod_base = OTHER; note = thymine
  modified_base 158: mod_base = OTHER; note = thymine
  modified_base 160: mod_base = OTHER; note = thymine
  modified_base 167..169: mod_base = OTHER; note = thymine
  modified_base 172..173: mod_base = OTHER; note = thymine
  modified_base 187..188: mod_base = OTHER; note = thymine
  modified_base 195: mod_base = OTHER; note = thymine
  modified_base 198: mod_base = OTHER; note = thymine
  modified_base 200: mod_base = OTHER; note = thymine
  modified_base 214: mod_base = OTHER; note = thymine
  modified_base 217..218: mod_base = OTHER; note = thymine
  modified_base 220: mod_base = OTHER; note = thymine
  modified_base 222: mod_base = OTHER; note = thymine
  modified_base 233: mod_base = OTHER; note = thymine
  modified_base 239: mod_base = OTHER; note = thymine
  modified_base 242: mod_base = OTHER; note = thymine
  modified_base 246: mod_base = OTHER; note = thymine
  modified_base 251..252: mod_base = OTHER; note = thymine
  modified_base 255: mod_base = OTHER; note = thymine
  modified_base 258: mod_base = OTHER; note = thymine
  modified_base 260..261: mod_base = OTHER; note = thymine
  modified_base 272: mod_base = OTHER; note = thymine
  modified_base 275: mod_base = OTHER; note = thymine
  modified_base 279: mod_base = OTHER; note = thymine
  modified_base 284..285: mod_base = OTHER; note = thymine
  modified_base 287: mod_base = OTHER; note = thymine
  modified_base 289..290: mod_base = OTHER; note = thymine
  modified_base 297: mod_base = OTHER; note = thymine
  modified_base 300: mod_base = OTHER; note = thymine
  modified_base 306: mod_base = OTHER; note = thymine
  modified_base 309..310: mod_base = OTHER; note = thymine
  modified_base 324..325: mod_base = OTHER; note = thymine
  modified_base 329..330: mod_base = OTHER; note = thymine
  modified_base 336: mod_base = OTHER; note = thymine
  modified_base 338: mod_base = OTHER; note = thymine
  modified_base 340: mod_base = OTHER; note = thymine
  modified_base 351..352: mod_base = OTHER; note = thymine
  modified_base 357: mod_base = OTHER; note = thymine
  modified_base 361: mod_base = OTHER; note = thymine
  modified_base 365: mod_base = OTHER; note = thymine
  modified_base 374: mod_base = OTHER; note = thymine
  modified_base 378: mod_base = OTHER; note = thymine
  modified_base 383: mod_base = OTHER; note = thymine
  modified_base 395..396: mod_base = OTHER; note = thymine
  modified_base 407: mod_base = OTHER; note = thymine
  modified_base 409..410: mod_base = OTHER; note = thymine
  modified_base 413: mod_base = OTHER; note = thymine
  modified_base 416: mod_base = OTHER; note = thymine
  modified_base 418..419: mod_base = OTHER; note = thymine
  modified_base 422..423: mod_base = OTHER; note = thymine
  modified_base 429: mod_base = OTHER; note = thymine
  modified_base 431: mod_base = OTHER; note = thymine
  modified_base 434: mod_base = OTHER; note = thymine
  modified_base 447: mod_base = OTHER; note = thymine
  modified_base 449: mod_base = OTHER; note = thymine
  modified_base 457: mod_base = OTHER; note = thymine
  modified_base 467: mod_base = OTHER; note = thymine
  modified_base 469..470: mod_base = OTHER; note = thymine
  modified_base 474: mod_base = OTHER; note = thymine
  modified_base 478..480: mod_base = OTHER; note = thymine
  modified_base 486..487: mod_base = OTHER; note = thymine
  modified_base 492: mod_base = OTHER; note = thymine
  modified_base 507: mod_base = OTHER; note = thymine
  modified_base 510: mod_base = OTHER; note = thymine
  modified_base 514: mod_base = OTHER; note = thymine
  modified_base 516: mod_base = OTHER; note = thymine
  modified_base 520: mod_base = OTHER; note = thymine
  modified_base 529: mod_base = OTHER; note = thymine
  modified_base 532: mod_base = OTHER; note = thymine
  modified_base 540..541: mod_base = OTHER; note = thymine
  modified_base 546..547: mod_base = OTHER; note = thymine
  modified_base 570..572: mod_base = OTHER; note = thymine
  modified_base 575: mod_base = OTHER; note = thymine
  modified_base 583: mod_base = OTHER; note = thymine
  modified_base 585: mod_base = OTHER; note = thymine
  modified_base 587: mod_base = OTHER; note = thymine
  modified_base 589: mod_base = OTHER; note = thymine
  modified_base 591: mod_base = OTHER; note = thymine
  modified_base 593: mod_base = OTHER; note = thymine
  modified_base 603..604: mod_base = OTHER; note = thymine
  modified_base 611: mod_base = OTHER; note = thymine
  modified_base 620: mod_base = OTHER; note = thymine
  modified_base 626: mod_base = OTHER; note = thymine
  modified_base 632: mod_base = OTHER; note = thymine
  modified_base 640..641: mod_base = OTHER; note = thymine
  modified_base 644..645: mod_base = OTHER; note = thymine
  modified_base 655..656: mod_base = OTHER; note = thymine
  modified_base 682: mod_base = OTHER; note = thymine
  modified_base 691..692: mod_base = OTHER; note = thymine
  modified_base 695: mod_base = OTHER; note = thymine
  modified_base 697: mod_base = OTHER; note = thymine
  modified_base 699: mod_base = OTHER; note = thymine
  modified_base 703: mod_base = OTHER; note = thymine
  modified_base 706..708: mod_base = OTHER; note = thymine
  modified_base 717: mod_base = OTHER; note = thymine
  modified_base 728: mod_base = OTHER; note = thymine
  modified_base 730: mod_base = OTHER; note = thymine
  modified_base 743: mod_base = OTHER; note = thymine
  modified_base 752: mod_base = OTHER; note = thymine
  modified_base 763: mod_base = OTHER; note = thymine
  modified_base 777: mod_base = OTHER; note = thymine
  modified_base 779: mod_base = OTHER; note = thymine
  modified_base 788..790: mod_base = OTHER; note = thymine
  modified_base 792: mod_base = OTHER; note = thymine
  modified_base 800: mod_base = OTHER; note = thymine
  modified_base 816: mod_base = OTHER; note = thymine
  modified_base 831..832: mod_base = OTHER; note = thymine
  modified_base 834: mod_base = OTHER; note = thymine
  modified_base 839: mod_base = OTHER; note = thymine
  modified_base 841..844: mod_base = OTHER; note = thymine
  modified_base 854: mod_base = OTHER; note = thymine
  modified_base 856..857: mod_base = OTHER; note = thymine
  modified_base 864: mod_base = OTHER; note = thymine
  modified_base 867: mod_base = OTHER; note = thymine
  modified_base 878: mod_base = OTHER; note = thymine
  modified_base 881..882: mod_base = OTHER; note = thymine
  modified_base 887..891: mod_base = OTHER; note = thymine
  modified_base 894: mod_base = OTHER; note = thymine
  modified_base 899: mod_base = OTHER; note = thymine
  modified_base 902: mod_base = OTHER; note = thymine
  modified_base 906: mod_base = OTHER; note = thymine
  modified_base 914..915: mod_base = OTHER; note = thymine
  modified_base 917: mod_base = OTHER; note = thymine
  modified_base 920: mod_base = OTHER; note = thymine
  modified_base 924: mod_base = OTHER; note = thymine
  modified_base 935: mod_base = OTHER; note = thymine
  modified_base 942: mod_base = OTHER; note = thymine
  modified_base 951: mod_base = OTHER; note = thymine
  modified_base 957..959: mod_base = OTHER; note = thymine
  modified_base 961: mod_base = OTHER; note = thymine
  modified_base 966: mod_base = OTHER; note = thymine
  modified_base 970..973: mod_base = OTHER; note = thymine
  modified_base 977: mod_base = OTHER; note = thymine
  modified_base 980..981: mod_base = OTHER; note = thymine
  modified_base 996: mod_base = OTHER; note = thymine
  modified_base 998: mod_base = OTHER; note = thymine
  modified_base 1000: mod_base = OTHER; note = thymine
  modified_base 1002: mod_base = OTHER; note = thymine
  modified_base 1010: mod_base = OTHER; note = thymine
  modified_base 1014..1015: mod_base = OTHER; note = thymine
  modified_base 1025: mod_base = OTHER; note = thymine
  modified_base 1028: mod_base = OTHER; note = thymine
  modified_base 1035: mod_base = OTHER; note = thymine
  modified_base 1037: mod_base = OTHER; note = thymine
  modified_base 1040: mod_base = OTHER; note = thymine
  modified_base 1048: mod_base = OTHER; note = thymine
  modified_base 1053..1055: mod_base = OTHER; note = thymine
  modified_base 1064: mod_base = OTHER; note = thymine
  modified_base 1071: mod_base = OTHER; note = thymine
  modified_base 1073: mod_base = OTHER; note = thymine
  modified_base 1075: mod_base = OTHER; note = thymine
  modified_base 1083: mod_base = OTHER; note = thymine
  modified_base 1102: mod_base = OTHER; note = thymine
  modified_base 1106: mod_base = OTHER; note = thymine
  modified_base 1108: mod_base = OTHER; note = thymine
  modified_base 1114..1115: mod_base = OTHER; note = thymine
  modified_base 1126: mod_base = OTHER; note = thymine
  modified_base 1128: mod_base = OTHER; note = thymine
  modified_base 1135..1136: mod_base = OTHER; note = thymine
  modified_base 1147: mod_base = OTHER; note = thymine
  modified_base 1151: mod_base = OTHER; note = thymine
  modified_base 1169..1170: mod_base = OTHER; note = thymine
  modified_base 1185: mod_base = OTHER; note = thymine
  modified_base 1190: mod_base = OTHER; note = thymine
  modified_base 1195: mod_base = OTHER; note = thymine
  modified_base 1198..1200: mod_base = OTHER; note = thymine
  modified_base 1204: mod_base = OTHER; note = thymine
  modified_base 1208..1210: mod_base = OTHER; note = thymine
  modified_base 1212: mod_base = OTHER; note = thymine
  modified_base 1215: mod_base = OTHER; note = thymine
  modified_base 1220: mod_base = OTHER; note = thymine
  modified_base 1224..1225: mod_base = OTHER; note = thymine
  source 1..1227: mol_type = mRNA
atgttgtctacgaaacttttctggacgagttcggttttggcttccagtgcggtgcttgcctttcccacgtcgcctgcgactaggactcctcgcagcacttccattctgaaggttgccgtggaccccacaacagtgaccaagaaggaatacgaagacatctgcggggtttcgttcgacgccgacacgttggaagatcgtctaaaggcaacgaactacttgtatcccaaacacgtcgaagtcatcgatgacattgctcctattgccggagccatggtggatgaaattctcttggaaactggtgaaaatgcttggcagccgcaagattaccttcccgatctctcgcaggacaattggcatgactccatcaaggaagtccgtgccatggccaaggaaattccggacgaactcttggtcgtgttgattggcgatatggtgacggaagaagctctcccaacctaccagacgctcttgaatacctttgaaggttgcgatgaccccaccggcactagtgaatctccctgggcacgctggtcccgcggttggacttcggaagaaaaccggcacggcgatttgctcaacaaatatctctatctgggcggccgttgcgacatgcgcaacatcgaggtcaccatccagcacttgattacgaacggattcaacccgcaagcccgcaaagacccctaccgcggattcgtgtatacgtcctttcaggaacgtgcgaccaaaatctcgcacggcaacgtgggcaagctggcccgcacgtacggcgaaaagaatctgaacaagatttgtgcaaagatcgcgggcgacgaaggtcgccacgagaaggcttatcaaatcttttccgaagaaatcttaaagcgtgatccggacggactcattcacgtttttggtgacatgatgcgtggacagattgtcatgcctgcggaacagatgacggatggcaaagatccggatttgtacgataacttttccatggttgcccagaagacgggtgtgtatacagccctcgattacgcagaaatcatcgaccatctggtgaaacggtgggatttggagcacctggaaggtctgtcaccggctgccgaaaaggaacgggagtacctgtgccgattgccggaacgctatcgcaaattggcgacccgatcgatgaacaaaaagaagaaagttaccgaagacgaggatccactcaaatcctttggctggatttatggtcggatggcttga

SEQ ID NO: 3 - DNA, 2261 bp, Nannochloropsis gaditana
  source 1..2261: mol_type = genomic DNA
atgcacgtctacccagacaaagttgaggtaattaccagcctggagcccatggtagagggtatgcttaccaagctgcttcttcccaccggagaaaaagcgtggcagccgcaggacttcgtgccagacctgacgaaggagggttgggaggaggaggtcaaggagttccgcgaaaaagccgccaccctccccgacgagactttggtagtgcttgtcggagacatggtgacggaggaagccctaccaacttaccagaccttgttgaacaccttcgagggcgtcgacgatcctactggcacctcaccatcggcttggtgcaagtggacccggggttggacctcggaagagaaccgccatggtgacttgctgaacagatacctgtatttgacggggcgagtggacatgcgctctattgagtgcaccatccagcaccttgtaagtgtccgtgaagagatgaggatgagggaaagcgcgtagcgtacgcagtgaaaggttgcaattgcatgggaggagagcccaaaggactcaaaacgaaagtcttgcgtaagcgggaagatagtgatctgatattgttattattgaccatttttatgtacacacagatttctagcggatttaatcccaagatcaagaaagacccttacaagggcttcgtctacacctcttttcaggagcgagccaccaagatttctcaccagaatgtcgcacgtcttgcgaacaatgcaggagatggtaaccttggccttatctgcagcaagattgcgggggacgagtcacgacacgagaaggcgtaccaggccattttcaaggagatcttgaacagagacccggagggaggccttaagtcgttctacgagttgatgacagaccaggtaaaaactagatcttgaaccgtgctagcgtaattcgacgtggttgacttttgtacttaggaaccttcacctaacttcacccccccttagatcaccatgcccgccgtaatgatgacagacgggcacgatccagacctgttcgagcacttttccatcactgctcagaagttaggtgtctacacggccgtggattacgcaaatatcttggatcacctcgtggaaatctgggatatcgagcacattggaggcctgtctggcgagggggcacgttatcaagaaaagctttgcaagctcggcccgcggtacctgaaactggctgagatgagcttagatcgtgtcaagaagatgccgaagactccttattcttggattcatggccgcatggcgtagatgcacgtctacccagacaaagttgaggtaattaccagcctggagcccatggtagagggtatgcttaccaagctgcttcttcccaccggagaaaaagcgtggcagccgcaggacttcgtgccagacctgacgaaggagggttgggaggaggaggtcaaggagttccgcgaaaaagccgccaccctccccgacgagactttggtagtgcttgtcggagacatggtgacggaggaagccctaccaacttaccagaccttgttgaacaccttcgagggcgtcgacgatcctactggcacctcaccatcggcttggtgcaagtggacccggggttggacctcggaagagaaccgccatggtgacttgctgaacagatacctgtatttgacggggcgagtggacatgcgctctattgagtgcaccatccagcaccttatttctagcggatttaatcccaagatcaagaaagacccttacaagggcttcgtctacacctcttttcaggagcgagccaccaagatttctcaccagaatgtcgcacgtcttgcgaacaatgcaggagatggtaaccttggccttatctgcagcaagattgcgggggacgagtcacgacacgagaaggcgtaccaggccattttcaaggagatcttgaacagagacccggagggaggccttaagtcgttctacgagttgatgacagaccagatcaccatgcccgccgtaatgatgacagacgggcacgatccagacctgttcgagcacttttccatcactgctcagaagttaggtgtctacacggccgtggattacgcaaatatcttggatcacctcgtggaaatctgggatatcgagcacattggaggcctgtctggcgagggggcacgttatcaagaaaagctttgcaagctcggcccgcggtacctgaaactggctgagatgagcttagatcgtgtcaagaagatgccgaagactccttattcttggattcatggccgcatggcgtag

SEQ ID NO: 4 - DNA, 1263 bp, Nannochloropsis salina
  source 1..1263: mol_type = genomic DNA
atgcacgtctacccagacaaagttgaggtaattaccagcctggagcccatggtagagggtatgcttaccaagctgcttcttcccactggagaaaaagcgtggcagccacaggacttcgtgccagacctgacgaaggagggttgggaggaggaggtcaaggagttccgcgaaaaagccgccaccctccccgacgagactctggtagtgcttgtcggagacatggtgacggaggaagccctaccaacttaccagaccttgttgaacaccttcgagggcgtggacgatcctactggcacctcaccatcggcttggtgcaagtggacccggggttggacctcggaagagaaccgccatggtgacttgctgaacagatacctgtacttgacggggcgagtggacatgcgctctgttgagtgcaccatccagcaccttgtaagtgtccgtgaagagatgaggatgagggaaagcgcgtagcgtacgcagggagatgttgcaattgtatggaaggagagtccatttgactcgaaacgaaagtcttgcgtaagcaactcggaagatagtgatctgatgtggttatgactgaccattattgtgtacacacagatttctagcggattcaaccccaagatcaagaaagacccttataagggcttcgtctacacctcttttcaggagcgagccaccaagatttctcaccagaatgtcgcacgtcttgcgaacaatgcaggagatggtaaccttggccttatctgcagcaagattgcgggggacgagtcgcgacacgagaaggcgtaccaggccattttcaaggagatcttgaacagagacccggagggaggccttaagtcgttctacgagttgatgacagaccaggtaaaaactagatcttgacccgtgctagcgtgattcgacgtggttgacttctgtacttaggaaccttcacctcacttcacctccccttagatcaccatgcccgccgtaatgatgacagacgggcacgatccagacctgttcgagcacttttccatcactgctcagaagctaggtgtctacacggccgtggattacgcaaatatcttggatcacctggtggaaatctgggatatcgagcacattggaggcctgtctggcgagggtgcacgctatcaagaaaagctttgcaagctcggcccgcggtatttgaaactggctgagatgagcttagatcgtgtcaagaagatgccgaagactccttattcttggattcatggccgcatggcgtag

SEQ ID NO: 5 - DNA, 1302 bp, Nannochloropsis oceanica
  source 1..1302: mol_type = genomic DNA
atgcacgtctaccccgacaagcacgaagtcattgtcagcatggagcccatggtggaagggatgctctcgaagctcctcctccccacgggggagaaggcttggcaaccgtccgactttgtcccggacctgaccaaggaagggtgggaagacgaggtaaaggaattcagggaaaaggcggagacgttgccggacgagacgctggtggtcttggtgggggacatggtgactgaggaggcgttgcccacttaccagacgctgttgaacacgttcgagggcgtggatgaccccaccgggacgagtccttcggcctggtgccgatggacacgggggtggacgagcgaggagaacaggcacggggatctgctcaaccggtacttgtacttgacggggcgcgtggacatgcgcgcgatcgagtgcacgattcagcacctggtacgtggggatgggagggagggagagagggagggatggaggaaggctcctgttcttgtgttcccccccttcgtctcgcgcgatgtctaactcattcttcccaccctccctccctctgcgtccgtaaattagatttcgagcggtttcaaccccaaaatcaagaaggatccgtacaagggtttcgtatacacctccttccaagagcgagcaaccaagatctcccatcagaatgtggcgcggctggccaacagcgccggtgatggcaatctgggtctgatctgcagtaagattgcaggggatgagtcgaggcacgagaaggcataccaagccatcttccaggagatcctcaagaaggacccggaaggggggttgaagagcttctatgagttaatgagtgatcaggtagggagggcggagggagagggaggggggaaagggagggaggtaggtacgcattaggaggaagggaacgacacaagtactttcaccatgggccaagatacggctgtttacctttccctcctccctccctctctccccctccctccctccctccctccctcatctcagatcaccatgcctgccgtgatgatgacggacggccacgacccggacctcttcgagcacttctcggtcacggcgcagaagctcggtgtctacacggcagtggactacgccaacattctggaatatttggtggagacatgggacatcgagcacatgggaggcctgtcttctgaaggggccaagtaccaggagaagctctgcaagctcgcgccccgatacatgaggcttgccgagatgagtcttgaccgggtgaaaaagatgcccaagacgccttattcttggatccatggacgcgtggcttaa

SEQ ID NO: 6 - RNA, 939 nt, Thalassiosira pseudonana
  modified_base 2: mod_base = OTHER; note = thymine
  modified_base 19: mod_base = OTHER; note = thymine
  modified_base 22: mod_base = OTHER; note = thymine
  modified_base 37: mod_base = OTHER; note = thymine
  modified_base 41: mod_base = OTHER; note = thymine
  modified_base 50: mod_base = OTHER; note = thymine
  modified_base 52: mod_base = OTHER; note = thymine
  modified_base 67: mod_base = OTHER; note = thymine
  modified_base 71: mod_base = OTHER; note = thymine
  modified_base 80: mod_base = OTHER; note = thymine
  modified_base 85: mod_base = OTHER; note = thymine
  modified_base 89: mod_base = OTHER; note = thymine
  modified_base 98: mod_base = OTHER; note = thymine
  modified_base 110: mod_base = OTHER; note = thymine
  modified_base 122: mod_base = OTHER; note = thymine
  modified_base 124..125: mod_base = OTHER; note = thymine
  modified_base 128: mod_base = OTHER; note = thymine
  modified_base 131: mod_base = OTHER; note = thymine
  modified_base 134: mod_base = OTHER; note = thymine
  modified_base 137: mod_base = OTHER; note = thymine
  modified_base 146: mod_base = OTHER; note = thymine
  modified_base 149: mod_base = OTHER; note = thymine
  modified_base 164: mod_base = OTHER; note = thymine
  modified_base 172: mod_base = OTHER; note = thymine
  modified_base 182: mod_base = OTHER; note = thymine
  modified_base 185: mod_base = OTHER; note = thymine
  modified_base 193..194: mod_base = OTHER; note = thymine
  modified_base 202: mod_base = OTHER; note = thymine
  modified_base 210: mod_base = OTHER; note = thymine
  modified_base 215..216: mod_base = OTHER; note = thymine
  modified_base 222: mod_base = OTHER; note = thymine
  modified_base 231: mod_base = OTHER; note = thymine
  modified_base 235: mod_base = OTHER; note = thymine
  modified_base 244: mod_base = OTHER; note = thymine
  modified_base 247: mod_base = OTHER; note = thymine
  modified_base 256: mod_base = OTHER; note = thymine
  modified_base 261..262: mod_base = OTHER; note = thymine
  modified_base 276: mod_base = OTHER; note = thymine
  modified_base 287: mod_base = OTHER; note = thymine
  modified_base 290: mod_base = OTHER; note = thymine
  modified_base 298: mod_base = OTHER; note = thymine
  modified_base 300: mod_base = OTHER; note = thymine
  modified_base 302: mod_base = OTHER; note = thymine
  modified_base 304: mod_base = OTHER; note = thymine
  modified_base 308: mod_base = OTHER; note = thymine
  modified_base 310: mod_base = OTHER; note = thymine
  modified_base 312: mod_base = OTHER; note = thymine
  modified_base 319: mod_base = OTHER; note = thymine
  modified_base 326: mod_base = OTHER; note = thymine
  modified_base 331: mod_base = OTHER; note = thymine
  modified_base 335: mod_base = OTHER; note = thymine
  modified_base 341: mod_base = OTHER; note = thymine
  modified_base 347..348: mod_base = OTHER; note = thymine
  modified_base 356: mod_base = OTHER; note = thymine
  modified_base 359..360: mod_base = OTHER; note = thymine
  modified_base 366: mod_base = OTHER; note = thymine
  modified_base 370..371: mod_base = OTHER; note = thymine
  modified_base 381: mod_base = OTHER; note = thymine
  modified_base 397: mod_base = OTHER; note = thymine
  modified_base 402: mod_base = OTHER; note = thymine
  modified_base 406..407: mod_base = OTHER; note = thymine
  modified_base 410: mod_base = OTHER; note = thymine
  modified_base 412: mod_base = OTHER; note = thymine
  modified_base 417..418: mod_base = OTHER; note = thymine
  modified_base 421..423: mod_base = OTHER; note = thymine
  modified_base 435: mod_base = OTHER; note = thymine
  modified_base 443: mod_base = OTHER; note = thymine
  modified_base 445: mod_base = OTHER; note = thymine
  modified_base 447: mod_base = OTHER; note = thymine
  modified_base 456: mod_base = OTHER; note = thymine
  modified_base 458: mod_base = OTHER; note = thymine
  modified_base 467: mod_base = OTHER; note = thymine
  modified_base 478: mod_base = OTHER; note = thymine
  modified_base 494: mod_base = OTHER; note = thymine
  modified_base 503..505: mod_base = OTHER; note = thymine
  modified_base 507: mod_base = OTHER; note = thymine
  modified_base 515..516: mod_base = OTHER; note = thymine
  modified_base 525: mod_base = OTHER; note = thymine
  modified_base 534: mod_base = OTHER; note = thymine
  modified_base 537: mod_base = OTHER; note = thymine
  modified_base 547: mod_base = OTHER; note = thymine
  modified_base 556..559: mod_base = OTHER; note = thymine
  modified_base 561: mod_base = OTHER; note = thymine
  modified_base 569..570: mod_base = OTHER; note = thymine
  modified_base 572: mod_base = OTHER; note = thymine
  modified_base 582: mod_base = OTHER; note = thymine
  modified_base 588: mod_base = OTHER; note = thymine
  modified_base 592..593: mod_base = OTHER; note = thymine
  modified_base 595..596: mod_base = OTHER; note = thymine
  modified_base 599: mod_base = OTHER; note = thymine
  modified_base 604..606: mod_base = OTHER; note = thymine
  modified_base 613..614: mod_base = OTHER; note = thymine
  modified_base 617: mod_base = OTHER; note = thymine
  modified_base 629..630: mod_base = OTHER; note = thymine
  modified_base 632: mod_base = OTHER; note = thymine
  modified_base 635: mod_base = OTHER; note = thymine
  modified_base 646..647: mod_base = OTHER; note = thymine
  modified_base 650: mod_base = OTHER; note = thymine
  modified_base 657: mod_base = OTHER; note = thymine
  modified_base 666: mod_base = OTHER; note = thymine
  modified_base 673..674: mod_base = OTHER; note = thymine
  modified_base 676: mod_base = OTHER; note = thymine
  modified_base 678: mod_base = OTHER; note = thymine
  modified_base 685..687: mod_base = OTHER; note = thymine
  modified_base 690: mod_base = OTHER; note = thymine
  modified_base 693: mod_base = OTHER; note = thymine
  modified_base 695: mod_base = OTHER; note = thymine
  modified_base 705: mod_base = OTHER; note = thymine
  modified_base 707..708: mod_base = OTHER; note = thymine
  modified_base 713..715: mod_base = OTHER; note = thymine
  modified_base 717: mod_base = OTHER; note = thymine
  modified_base 724..725: mod_base = OTHER; note = thymine
  modified_base 729..730: mod_base = OTHER; note = thymine
  modified_base 732: mod_base = OTHER; note = thymine
  modified_base 738: mod_base = OTHER; note = thymine
  modified_base 740..741: mod_base = OTHER; note = thymine
  modified_base 743..744: mod_base = OTHER; note = thymine
  modified_base 750: mod_base = OTHER; note = thymine
  modified_base 752..753: mod_base = OTHER; note = thymine
  modified_base 755: mod_base = OTHER; note = thymine
  modified_base 759: mod_base = OTHER; note = thymine
  modified_base 762..763: mod_base = OTHER; note = thymine
  modified_base 770..771: mod_base = OTHER; note = thymine
  modified_base 779: mod_base = OTHER; note = thymine
  modified_base 783: mod_base = OTHER; note = thymine
  modified_base 787..788: mod_base = OTHER; note = thymine
  modified_base 792: mod_base = OTHER; note = thymine
  modified_base 817: mod_base = OTHER; note = thymine
  modified_base 819: mod_base = OTHER; note = thymine
  modified_base 821..823: mod_base = OTHER; note = thymine
  modified_base 828..830: mod_base = OTHER; note = thymine
  modified_base 834: mod_base = OTHER; note = thymine
  modified_base 841: mod_base = OTHER; note = thymine
  modified_base 843: mod_base = OTHER; note = thymine
  modified_base 846: mod_base = OTHER; note = thymine
  modified_base 850..851: mod_base = OTHER; note = thymine
  modified_base 864: mod_base = OTHER; note = thymine
  modified_base 866: mod_base = OTHER; note = thymine
  modified_base 870: mod_base = OTHER; note = thymine
  modified_base 885: mod_base = OTHER; note = thymine
  modified_base 888: mod_base = OTHER; note = thymine
  modified_base 890: mod_base = OTHER; note = thymine
  modified_base 894..895: mod_base = OTHER; note = thymine
  modified_base 897: mod_base = OTHER; note = thymine
  modified_base 899: mod_base = OTHER; note = thymine
  modified_base 903: mod_base = OTHER; note = thymine
  modified_base 910..912: mod_base = OTHER; note = thymine
  modified_base 916: mod_base = OTHER; note = thymine
  modified_base 920..923: mod_base = OTHER; note = thymine
  modified_base 927: mod_base = OTHER; note = thymine
  modified_base 937: mod_base = OTHER; note = thymine
  source 1..939: mol_type = mRNA
ctcgaaaccggagagaaatcatggcaaccccaagactacctccccgacctctccaaccaagaagcctacatcgacgacatcaagtccctccgcgaaatggccgccggcgtccccgacgacgtcttcgtcgtcctcgtgggagacatgatcaccgaagaggcactcccaacctaccaaaccctcctcaacacgttcgaggggtgcgacgatcccattggcactacggagagtgcatgggcacgatggtcacgggggtggacttcggaggagaaccgtcacggagacctcctcaacaaatatctctacctgtctggaaagtgcgacatgcgatccatcgaagtgacgattcaacacctcattaccaatgggttcaaccccggtgcgaggaaagacccctaccgtggcttcctgtacacttcgtttcaagaacgagctaccaaaatctctcacgggaatgtaggcaagctggccaaggactggggagccaaggacatgcaacgaatttgtgcaaagattgcgggggatgagggacgtcatgagaaggcataccaggccttttgtacggagattctaaagagggatccggatggattgttgatggagtttggggacttgatgagagggcagattgtcatgccggcggagttgatgacggatgggaaggataccaagttgtatgagaactttagtgctgtggcgcaacgtcttgaggtttataccgccttggattatgcggatattattgaacatcttgtggatcattggaagattggggagctgactggcttgagtagcgaggcggagaaggagcaggagtatatttgccgtttgcctcagaggtatcgtaagttggcggagaggagtatgaataaggcgaagaaggatggtgtggattatgtgcctcaagagtttggatggattttcaatcgcaaggcgtag

SEQ ID NO: 7 - RNA, 1330 nt, Chlorella variabilis
  modified_base 6: mod_base = OTHER; note = thymine
  modified_base 14: mod_base = OTHER; note = thymine
  modified_base 18: mod_base = OTHER; note = thymine
  modified_base 22: mod_base = OTHER; note = thymine
  modified_base 30: mod_base = OTHER; note = thymine
  modified_base 33: mod_base = OTHER; note = thymine
  modified_base 37: mod_base = OTHER; note = thymine
  modified_base 41: mod_base = OTHER; note = thymine
  modified_base 46: mod_base = OTHER; note = thymine
  modified_base 49: mod_base = OTHER; note = thymine
  modified_base 51: mod_base = OTHER; note = thymine
  modified_base 59: mod_base = OTHER; note = thymine
  modified_base 64: mod_base = OTHER; note = thymine
  modified_base 81..83: mod_base = OTHER; note = thymine
  modified_base 92: mod_base = OTHER; note = thymine
  modified_base 105..107: mod_base = OTHER; note = thymine
  modified_base 114: mod_base = OTHER; note = thymine
  modified_base 117: mod_base = OTHER; note = thymine
  modified_base 123: mod_base = OTHER; note = thymine
  modified_base 125: mod_base = OTHER; note = thymine
  modified_base 135: mod_base = OTHER; note = thymine
  modified_base 141: mod_base = OTHER; note = thymine
  modified_base 148: mod_base = OTHER; note = thymine
  modified_base 162..163: mod_base = OTHER; note = thymine
  modified_base 167: mod_base = OTHER; note = thymine
  modified_base 170: mod_base = OTHER; note = thymine
  modified_base 175: mod_base = OTHER; note = thymine
  modified_base 180: mod_base = OTHER; note = thymine
  modified_base 194: mod_base = OTHER; note = thymine
  modified_base 205: mod_base = OTHER; note = thymine
  modified_base 214: mod_base = OTHER; note = thymine
  modified_base 225: mod_base = OTHER; note = thymine
  modified_base 228: mod_base = OTHER; note = thymine
  modified_base 243: mod_base = OTHER; note = thymine
  modified_base 282: mod_base = OTHER; note = thymine
  modified_base 285: mod_base = OTHER; note = thymine
  modified_base 297: mod_base = OTHER; note = thymine
  modified_base 300: mod_base = OTHER; note = thymine
  modified_base 324: mod_base = OTHER; note = thymine
  modified_base 328: mod_base = OTHER; note = thymine
  modified_base 330: mod_base = OTHER; note = thymine
  modified_base 333: mod_base = OTHER; note = thymine
  modified_base 342: mod_base = OTHER; note = thymine
  modified_base 350: mod_base = OTHER; note = thymine
  modified_base 352: mod_base = OTHER; note = thymine
  modified_base 364: mod_base = OTHER; note = thymine
  modified_base 366: mod_base = OTHER; note = thymine
  modified_base 369: mod_base = OTHER; note = thymine
  modified_base 375: mod_base = OTHER; note = thymine
  modified_base 378: mod_base = OTHER; note = thymine
  modified_base 387: mod_base = OTHER; note = thymine
  modified_base 395: mod_base = OTHER; note = thymine
  modified_base 398: mod_base = OTHER; note = thymine
  modified_base 413..414: mod_base = OTHER; note = thymine
  modified_base 417: mod_base = OTHER; note = thymine
  modified_base 431: mod_base = OTHER; note = thymine
  modified_base 440..441: mod_base = OTHER; note = thymine
  modified_base 444: mod_base = OTHER; note = thymine
  modified_base 453: mod_base = OTHER; note = thymine
  modified_base 462: mod_base = OTHER; note = thymine
  modified_base 483: mod_base = OTHER; note = thymine
  modified_base 494: mod_base = OTHER; note = thymine
  modified_base 497..499: mod_base = OTHER; note = thymine
  modified_base 501: mod_base = OTHER; note = thymine
  modified_base 503: mod_base = OTHER; note = thymine
  modified_base 507: mod_base = OTHER; note = thymine
  modified_base 510: mod_base = OTHER; note = thymine
  modified_base 519: mod_base = OTHER; note = thymine
  modified_base 522: mod_base = OTHER; note = thymine
  modified_base 537: mod_base = OTHER; note = thymine
  modified_base 545: mod_base = OTHER; note = thymine
  modified_base 549: mod_base = OTHER; note = thymine
  modified_base 555: mod_base = OTHER; note = thymine
  modified_base 558: mod_base = OTHER; note = thymine
  modified_base 565: mod_base = OTHER; note = thymine
  modified_base 567: mod_base = OTHER; note = thymine
  modified_base 576: mod_base = OTHER; note = thymine
  modified_base 608: mod_base = OTHER; note = thymine
  modified_base 617: mod_base = OTHER; note = thymine
  modified_base 629: mod_base = OTHER; note = thymine
  modified_base 633: mod_base = OTHER; note = thymine
  modified_base 660: mod_base = OTHER; note = thymine
  modified_base 663: mod_base = OTHER; note = thymine
  modified_base 671: mod_base = OTHER; note = thymine
  modified_base 674: mod_base = OTHER; note = thymine
  modified_base 677: mod_base = OTHER; note = thymine
  modified_base 681: mod_base = OTHER; note = thymine
  modified_base 693: mod_base = OTHER; note = thymine
  modified_base 699: mod_base = OTHER; note = thymine
  modified_base 708: mod_base = OTHER; note = thymine
  modified_base 714: mod_base = OTHER; note = thymine
  modified_base 720: mod_base = OTHER; note = thymine
  modified_base 729: mod_base = OTHER; note = thymine
  modified_base 732: mod_base = OTHER; note = thymine
  modified_base 737: mod_base = OTHER; note = thymine
  modified_base 739: mod_base = OTHER; note = thymine
  modified_base 744: mod_base = OTHER; note = thymine
  modified_base 770: mod_base = OTHER; note = thymine
  modified_base 774: mod_base = OTHER; note = thymine
  modified_base 779..780: mod_base = OTHER; note = thymine
  modified_base 783: mod_base = OTHER; note = thymine
  modified_base 785: mod_base = OTHER; note = thymine
  modified_base 791: mod_base = OTHER; note = thymine
  modified_base 794..795: mod_base = OTHER; note = thymine
  modified_base 816: mod_base = OTHER; note = thymine
  modified_base 840: mod_base = OTHER; note = thymine
  modified_base 847: mod_base = OTHER; note = thymine
  modified_base 851: mod_base = OTHER; note = thymine
  modified_base 864: mod_base = OTHER; note = thymine
  modified_base 867: mod_base = OTHER; note = thymine
  modified_base 876: mod_base = OTHER; note = thymine
  modified_base 878: mod_base = OTHER; note = thymine
  modified_base 888: mod_base = OTHER; note = thymine
  modified_base 902: mod_base = OTHER; note = thymine
  modified_base 915..916: mod_base = OTHER; note = thymine
  modified_base 920: mod_base = OTHER; note = thymine
  modified_base 930: mod_base = OTHER; note = thymine
  modified_base 933: mod_base = OTHER; note = thymine
  modified_base 941..942: mod_base = OTHER; note = thymine
  modified_base 944..945: mod_base = OTHER; note = thymine
  modified_base 969: mod_base = OTHER; note = thymine
  modified_base 972: mod_base = OTHER; note = thymine
  modified_base 974: mod_base = OTHER; note = thymine
  modified_base 977..978: mod_base = OTHER; note = thymine
  modified_base 987: mod_base = OTHER; note = thymine
  modified_base 990: mod_base = OTHER; note = thymine
  modified_base 1002..1003: mod_base = OTHER; note = thymine
  modified_base 1005: mod_base = OTHER; note = thymine
  modified_base 1008: mod_base = OTHER; note = thymine
  modified_base 1020: mod_base = OTHER; note = thymine
  modified_base 1023: mod_base = OTHER; note = thymine
  modified_base 1044: mod_base = OTHER; note = thymine
  modified_base 1047: mod_base = OTHER; note = thymine
  modified_base 1065: mod_base = OTHER; note = thymine
  modified_base 1067..1068: mod_base = OTHER; note = thymine
  modified_base 1076..1077: mod_base = OTHER; note = thymine
  modified_base 1079: mod_base = OTHER; note = thymine
  modified_base 1086: mod_base = OTHER; note = thymine
  modified_base 1098: mod_base = OTHER; note = thymine
  modified_base 1104: mod_base = OTHER; note = thymine
  modified_base 1106: mod_base = OTHER; note = thymine
  modified_base 1121: mod_base = OTHER; note = thymine
  modified_base 1124: mod_base = OTHER; note = thymine
  modified_base 1131: mod_base = OTHER; note = thymine
  modified_base 1134: mod_base = OTHER; note = thymine
  modified_base 1143: mod_base = OTHER; note = thymine
  modified_base 1146: mod_base = OTHER; note = thymine
  modified_base 1154: mod_base = OTHER; note = thymine
  modified_base 1161..1162: mod_base = OTHER; note = thymine
  modified_base 1179: mod_base = OTHER; note = thymine
  modified_base 1208: mod_base = OTHER; note = thymine
  modified_base 1212: mod_base = OTHER; note = thymine
  modified_base 1215: mod_base = OTHER; note = thymine
  modified_base 1221: mod_base = OTHER; note = thymine
  modified_base 1232..1233: mod_base = OTHER; note = thymine
  modified_base 1242: mod_base = OTHER; note = thymine
  modified_base 1246: mod_base = OTHER; note = thymine
  modified_base 1268: mod_base = OTHER; note = thymine
  modified_base 1290: mod_base = OTHER; note = thymine
  modified_base 1295..1296: mod_base = OTHER; note = thymine
  modified_base 1301: mod_base = OTHER; note = thymine
  modified_base 1305: mod_base = OTHER; note = thymine
  modified_base 1307..1308: mod_base = OTHER; note = thymine
  modified_base 1320: mod_base = OTHER; note = thymine
  modified_base 1326: mod_base = OTHER; note = thymine
  modified_base 1328: mod_base = OTHER; note = thymine
  source 1..1330: mol_type = mRNA
gcccctggagagctccctggatagcacgctgctggctcggtgcgctcgtgtgcagcagtcccctccccgagccccgacactttcaggagcgtcaaggcaaaggctttgcagcatggtggcgctgtcggccggcgtgaacgtggcgcctagcagccagcacattgcatcgtgcggtgcgctgcggcagagccgctccaccgcggctccccgccgtgccgccgccgtggtgcgcccccgcgcggtggccgcccccgaggccgccgcgcgccagggccccaccatcgtgaacggccaggtgctgcacagcgccaccaaggagcagctggatgtggtggccagcatggacaagtatgcggaggagcatgtgctgcccatcctcaagcccgtggagaagtgctggcaggcccaggacttcctgccccaccccgagtccccagacttcctcgaccacgtgcaccagctgcagcagcgcaccaaggaggtcccagacgactactttgtgtgcctggtgggcgacatgatcacggaggaggcgctgcccacctacatggccatgctcaacactctggacggcgtgcgcgacgagacgggcgccgcgcccacgccctggggccgctggacgcggcagtgggtggcggaggagaaccggcacggcgacctgatgaacaagtactgctggctcagcgggcgcgtggacctgcgccaggtggaggtgaccatccagaacctcatcgggtctggcatggaccccaagaccgagaacaacccctacctgggcttcatctacacatccttccaggagcgcgccaccaaggtgagccacggcaacacggcgcgcatggcggctgagtacggcgacgaggtgctgggcaaggtgtgcggcgccatcgccagcgacgagtcgcgccacgagattgcctacacccgcatcgtggacgagttcttcaggcaggaccccaacaacgccatgctgtgcttcgccgacatgatgcgcaagcagattgtcatgcccgcgcacctcatggacgacggcaagcacggcatcctcaacggcggccgcaacctgttcaccgacttctccgaggtggcggagcgcctcggcgtctaccaggccagcgactactgcgacatcatggagcacctggtggcgcgctgggacattgagcaccgcgagggcctgcgcggggaggcggcagaggcccaggagtacctgatgaagctgcccacccgcttccgcaagctgagtgagcgggcagcggcccgcaagtccaaggcgcagcccgcgcgcgtgcagttcagctggatcttcaaccgcgaggtgaaggtgtga

SEQ ID NO: 8 - protein, 429 aa, Dunaliella salina
MKSLLWKKNRGAPAIEASNGARASTCAPSLACKSRQKRPPIGHRLTAGVSATSTPEAAVPTAQAAAPNGTGSATIYIPSQKTHSLHAQGVEVIDSLQDWAEQRLLRFLKPVQSSWQPQDLLPEPSSPDFYDQVQELRRGAQQLPNDYLVVLVGDMVTEEALPSNMNLLNTLDCAKDETGASTSPWARWTRQWTAEENRHGDVLNKYLYLSGCVDMHSIENTIQRLIGSGLDPKLENNPYLCFVYTSFQERATKISHGNTARLAAYHGDTVLAKICGLVAADEARHEAAYTNIVEQLFIRDPEGAVLAFYDMMKKGIVMPAHLVDDGKHASNNGGASLFLDYATVADSTGVYTTADYADIVDHLVSRWRVADLRFPGGKAEEAQQFLCQHADRVRRLANIQAERKLRDRKRGKARIASFGWVNGRQVSLI

SEQ ID NO: 9 - protein, 402 aa, Dunaliella salina
MLGLQQKVVGAAGNGKQARCVKAAAGAEGRGVNSPLVTRAAATELPPRLKKPAPIILNNQVLHSITDERLDVVYSIADHVEKNVLPILTPVHKCWQPSELLPESSDPDFLDKVQDLRKRASSLPDDYLVVFTGDMITEEALPTYMTMLNTLDGVRDETGASMTPWGRWTRAWTAEENRHGDVMNKYMYLTGRVNMKAVEVTTQNLIGSGMDPKTENNPYLGFTYTSFQERATKTETATPNHTLANSRRCENLPMHLQTETATPNHTLANSRRIMDALFERDPNGAVQAFADMMKKQIVMPAHLMNDNEHDKKGRNLFADFSAVAERTKTYTAFDYADIMEHLVERWRVANLQGLRGEAAEAQEYLMKMPNRVRKLAERAYARRKANQHTDFSWVFNREVQLS

SEQ ID NO: 10 - RNA, 23 nt, unidentified
  modified_base 2: mod_base = OTHER; note = thymine
  modified_base 5: mod_base = OTHER; note = thymine
  modified_base 8..9: mod_base = OTHER; note = thymine
  modified_base 11: mod_base = OTHER; note = thymine
  modified_base 20: mod_base = OTHER; note = thymine
  modified_base 23: mod_base = OTHER; note = thymine
  misc_feature 1..23: note = guida
  source 1..23: mol_type = other RNA; note = synthetic RNA
gtagtgcttgtcggagacatggt

SEQ ID NO: 11 - RNA, 23 nt, unidentified
  modified_base 8: mod_base = OTHER; note = thymine
  modified_base 10..11: mod_base = OTHER; note = thymine
  misc_feature 1..23: note = guida
  source 1..23: mol_type = other RNA; note = synthetic RNA
aaggagatcttgaacagagaccc

SEQ ID NO: 12 - RNA, 23 nt, unidentified
  modified_base 1..2: mod_base = OTHER; note = thymine
  modified_base 5: mod_base = OTHER; note = thymine
  modified_base 10: mod_base = OTHER; note = thymine
  modified_base 17: mod_base = OTHER; note = thymine
  misc_feature 1..23: note = guida
  source 1..23: mol_type = other RNA; note = synthetic RNA
ttgataacgtgccccctcgccag

SEQ ID NO: 13 - DNA, 20 bp, unidentified
  misc_feature 1..20: note = Primer
  source 1..20: mol_type = other DNA; note = oligonucleotides
tcgaccgctccatgttgcaa

SEQ ID NO: 14 - DNA, 20 bp, unidentified
  misc_feature 1..20: note = Primer
  source 1..20: mol_type = other DNA; note = oligonucleotides
cttacaagggcttcgtctac

SEQ ID NO: 15 - DNA, 20 bp, unidentified
  misc_feature 1..20: note = Primer
  source 1..20: mol_type = other DNA; note = oligonucleotides
agcttttcttgataacgtgc

SEQ ID NO: 16 - DNA, 20 bp, unidentified
  misc_feature 1..20: note = Primer
  source 1..20: mol_type = other DNA; note = oligonucleotides
gtcatcgtcgtatccgagag

SEQ ID NO: 17 - DNA, 20 bp, unidentified
  misc_feature 1..20: note = Primer
  source 1..20: mol_type = other DNA; note = oligonucleotides
atgaaaaagcctgaactcac

SEQ ID NO: 18 - DNA, 20 bp, unidentified
  misc_feature 1..20: note = Primer
  source 1..20: mol_type = other DNA; note = oligonucleotides
ctattcctttgccctcggac

SEQ ID NO: 19 - DNA, 20 bp, unidentified
  misc_feature 1..20: note = Primer
  source 1..20: mol_type = other DNA; note = oligonucleotides
cttacaagggcttcgtctac

SEQ ID NO: 20 - DNA, 20 bp, unidentified
  misc_feature 1..20: note = Primer
  source 1..20: mol_type = other DNA; note = oligonucleotides
agcttttcttgataacgtgc

SEQ ID NO: 21 - protein, 333 aa, Nannochloropsis gaditana
MHVYPDKVEVITSLEPMVEGMLTKLLLPTGEKAWQPQDFVPDLTKEGWEEEVKEFREKAATLPDETLVVLVGDMVTEEALPTYQTLLNTFEGVDDPTGTSPSAWCKWTRGWTSEENRHGDLLNRYLYLTGRVDMRSIECTIQHLISSGFNPKIKKDPYKGFVYTSFQERATKISHQNVARLANNAGDGNLGLICSKIAGDESRHEKAYQAIFKEILNRDPEGGLKSFYELMTDQITMPAVMMTDGHDPDLFEHFSITAQKLGVYTAVDYANILDHLVEIWDIEHIGGLSGEGARYQEKLCKLGPRYLKLAEMSLDRVKKMPKTPYSWIHGRMA

SEQ ID NO: 22 - protein, 408 aa, Phaeodactylum tricornutum
MLSTKLFWTSSVLASSAVLAFPTSPATRTPRSTSILKVAVDPTTVTKKEYEDICGVSFDADTLEDRLKATNYLYPKHVEVIDDIAPIAGAMVDEILLETGENAWQPQDYLPDLSQDNWHDSIKEVRAMAKEIPDELLVVLIGDMVTEEALPTYQTLLNTFEGCDDPTGTSESPWARWSRGWTSEENRHGDLLNKYLYLGGRCDMRNIEVTIQHLITNGFNPQARKDPYRGFVYTSFQERATKISHGNVGKLARTYGEKNLNKICAKIAGDEGRHEKAYQIFSEEILKRDPDGLIHVFGDMMRGQIVMPAEQMTDGKDPDLYDNFSMVAQKTGVYTALDYAEIIDHLVKRWDLEHLEGLSPAAEKEREYLCRLPERYRKLATRSMNKKKKVTEDEDPLKSFGWIYGRMA

SEQ ID NO: 23 - protein, 333 aa, Nannochloropsis salina
MHVYPDKVEVITSLEPMVEGMLTKLLLPTGEKAWQPQDFVPDLTKEGWEEEVKEFREKAATLPDETLVVLVGDMVTEEALPTYQTLLNTFEGVDDPTGTSPSAWCKWTRGWTSEENRHGDLLNRYLYLTGRVDMRSVECTIQHLISSGFNPKIKKDPYKGFVYTSFQERATKISHQNVARLANNAGDGNLGLICSKIAGDESRHEKAYQAIFKEILNRDPEGGLKSFYELMTDQITMPAVMMTDGHDPDLFEHFSITAQKLGVYTAVDYANILDHLVEIWDIEHIGGLSGEGARYQEKLCKLGPRYLKLAEMSLDRVKKMPKTPYSWIHGRMA

SEQ ID NO: 24 - protein, 333 aa, Nannochloropsis oceanica
MHVYPDKHEVIVSMEPMVEGMLSKLLLPTGEKAWQPSDFVPDLTKEGWEDEVKEFREKAETLPDETLVVLVGDMVTEEALPTYQTLLNTFEGVDDPTGTSPSAWCRWTRGWTSEENRHGDLLNRYLYLTGRVDMRAIECTIQHLISSGFNPKIKKDPYKGFVYTSFQERATKISHQNVARLANSAGDGNLGLICSKIAGDESRHEKAYQAIFQEILKKDPEGGLKSFYELMSDQITMPAVMMTDGHDPDLFEHFSVTAQKLGVYTAVDYANILEYLVETWDIEHMGGLSSEGAKYQEKLCKLAPRYMRLAEMSLDRVKKMPKTPYSWIHGRVA

SEQ ID NO: 25 - protein, 312 aa, Thalassiosira pseudonana
LETGEKSWQPQDYLPDLSNQEAYIDDIKSLREMAAGVPDDVFVVLVGDMITEEALPTYQTLLNTFEGCDDPIGTTESAWARWSRGWTSEENRHGDLLNKYLYLSGKCDMRSIEVTIQHLITNGFNPGARKDPYRGFLYTSFQERATKISHGNVGKLAKDWGAKDMQRICAKIAGDEGRHEKAYQAFCTEILKRDPDGLLMEFGDLMRGQIVMPAELMTDGKDTKLYENFSAVAQRLEVYTALDYADIIEHLVDHWKIGELTGLSSEAEKEQEYICRLPQRYRKLAERSMNKAKKDGVDYVPQEFGWIFNRKA

SEQ ID NO: 26 - protein, 405 aa, Chlorella variabilis
MVALSAGVNVAPSSQHIASCGALRQSRSTAAPRRAAAVVRPRAVAAPEAAARQGPTIVNGQVLHSATKEQLDVVASMDKYAEEHVLPILKPVEKCWQAQDFLPHPESPDFLDHVHQLQQRTKEVPDDYFVCLVGDMITEEALPTYMAMLNTLDGVRDETGAAPTPWGRWTRQWVAEENRHGDLMNKYCWLSGRVDLRQVEVTIQNLIGSGMDPKTENNPYLGFIYTSFQERATKVSHGNTARMAAEYGDEVLGKVCGAIASDESRHEIAYTRIVDEFFRQDPNNAMLCFADMMRKQIVMPAHLMDDGKHGILNGGRNLFTDFSEVAERLGVYQASDYCDIMEHLVARWDIEHREGLRGEAAEAQEYLMKLPTRFRKLSERAAARKSKAQPARVQFSWIFNREVKV